(19) United States
(12) Patent Application Publication
(10) Pub. No.: US 2024/0347203 A1
(43) Pub. Date: Oct. 17, 2024

(54) Methods and Systems for Predicting Risk of Cardiovascular Disease

(71) Applicants: Taipei Veterans General Hospital, Taipei City (TW); Academia Sinica, Taipei City (TW); National Taiwan University, Taipei City (TW)

(72) Inventors: Hao-Min Cheng, Taipei City (TW); Yeong-Sung Lin, Taipei City (TW); Yennun Huang, Taipei City (TW); Chiu-Han Hsiao, Taipei City (TW); Po-Chun Yu, Taipei City (TW); Chia-Ying Hsieh, Taipei City (TW); Wei-Lun Chang, Taipei City (TW)

(73) Assignees: Taipei Veterans General Hospital, Taipei City (TW); Academia Sinica, Taipei City (TW); National Taiwan University, Taipei City (TW)

(21) Appl. No.: 18/299,051

(22) Filed: Apr. 12, 2023

(51) Int. Cl. G16H 50/30; G16B 20/00

(52) U.S. Cl. CPC G16H 50/30; G16B 20/00

(57) ABSTRACT

Methods and computer-implemented methods are used to predict a risk of cardiovascular disease by using a machine learning mode to analyze a relationship between the occurrence of cardiovascular disease and the health data of patients.

### FIELD OF THE INVENTION

The present invention relates to methods and computer-implemented methods which are used to predict a risk of cardiovascular disease by using a machine learning mode to analyze a relationship between the occurrence of cardiovascular disease and the health data of patients.

### BACKGROUND OF THE INVENTION

Hypertension is one of the most widespread public health problems in the world. Hypertension can increase the risk of serious health problems such as stroke, renal failure, and cardiovascular disease (CVD). Hypertension, if left untreated, can lead to heart attacks or strokes. In recent years, researchers have gained a greater understanding of the pathophysiology of hypertension, and more effective treatment and prevention methods have been developed. Among them, home blood pressure (HBP) levels have been proven that can predict future high blood pressure and cardiovascular.

Poorly controlled blood pressure is influenced by a variety of factors, including obesity, salt sensitivity, psychological stress, genetic predisposition, epigenetics (the regulation of gene expression without altering DNA sequence), sleep apnea, autonomic regulation, microbial communities, and environmental factors such as smoking, excessive salt intake, excessive alcohol consumption, and socioeconomic status. Often, these factors interact and affect various diseases in complicated ways. Because of this, conventional statistical models rarely reflect all the complex causal relationships between risk factors. Therefore, a comprehensive data analysis is essential for developing accurate disease prediction models. In the past, various statistical methods have been used to develop prediction models and discover important risk factors. However, artificial intelligence (AI) and big data have risen in prominence lately and are increasingly being used to develop disease prediction models.

### SUMMARY OF THE INVENTION

The present invention relates to methods and computer-implemented methods used to assess a subject for the prediction of risk of developing a cardiovascular disease event over a 1-month to 4-year period. These methods use a machine learning model to analyze a relationship between the occurrence of cardiovascular disease and the health data of the subject.

### BRIEF DESCRIPTION OF THE DRAWINGS

FIG. 1 shows the data source description of the present invention. n: numbers of patients.

FIG. 2 shows the healthcare system for estimating a risk of cardiovascular disease of a patient.

### DETAILED DESCRIPTION OF THE INVENTION

CVD prediction is an important goal in modern medicine. Predicting CVD risk to enable early treatment can significantly reduce the risk of serious illness. Blood pressure (BP) is a critical indicator that is measured in most health examinations. Previously, BP is only measured at hospitals or health centers; however, these measurements have become more accessible now, if lightweight BP monitoring devices that can be used at home have enabled regular BP monitoring. Based on the results shown in this paper, a relationship between CVD and BP is revealed clearly. The daily monitoring of BP, recording exercise, alcohol consumption, medication doses, and other data using wearable devices can facilitate the timely accurate treatment of CVD in the future.

Prediction of CVD can also be increased if blood pressure data of the patient before CVD occurs is available.

The term “a” or “an” as used herein is to describe elements and ingredients of the present invention. The term is used only for convenience and providing the basic concepts of the present invention. Furthermore, the description should be understood as comprising one or at least one, and unless otherwise explicitly indicated by the context, singular terms include pluralities and plural terms include the singular. When used in conjunction with the word “comprising” in a claim, the term “a” or “an” may mean one or more than one.

The term “or” as used herein may mean “and/or.”

The present invention provides a method for producing a predicting model for estimating a risk of cardiovascular disease (CVD), which comprises: (a) obtaining a dataset from one or more sources, wherein the dataset comprises health data of non-CVD patients and CVD patients and data related to CVD onset time of the CVD patients, and the health data comprise demographic data, personal habits data, disease data, treatment data, blood analysis data, and blood pressure data; (b) inputting the dataset to at least one machine learning model for training the at least one machine learning model to predict CVD occurrence; (c) assessing accuracy of the at least one machine learning model from the step (b) and selecting a first machine learning model from the at least one machine learning model when the accuracy of the first machine learning model is higher than a threshold value of accuracy; and (d) using the first machine learning model to produce the prediction model for estimating a risk of CVD at different time points.

In one embodiment, the one or more sources comprise Taiwan Consortium of Hypertension-associated Cardiac Disease dataset and Taiwan Health and Welfare Data Science Center database.

In some aspects, the non-CVD patients are the patients who do not have a primary diagnosis of CVD, and the CDV patients are the patients who receive a primary diagnosis of CVD.

In another embodiment, the CVD comprises myocardial infarction, stroke, heart failure, cardiovascular death or a combination thereof.

In one embodiment, the demographic data comprise age, gender, body mass index (BMI), and waist circumference.

In another embodiment, the personal habits data comprise smoking, alcohol-drinking, and exercising habits. In some aspects, the smoking habit and alcohol-drinking habit are collected monthly or annually, and the exercising habit is collected weekly.

In one embodiment, the disease data comprise hypertension (HT), diabetes mellitus (DM), hyperlipidemia (HL) or a combination thereof.

In another embodiment, the treatment data comprise uses of antihypertensive drugs, antidiabetic drugs, lipid-lowering drugs, aspirin or a combination thereof.

In one embodiment, the blood analysis data comprise glutamic oxaloacetic transaminase (GOT), glutamic pyruvic transaminase (GPT), blood glucose, glycated hemoglobin (such as HbAlc), cholesterol, low-density lipoprotein (LDL), high-density lipoprotein (HDL) or a combination thereof.

In one embodiment, the blood pressure data comprise data of systolic blood pressure (SBP) and data of diastolic blood pressure (DBP). In a preferred embodiment, the data of SBP comprise the mean value of SBP, standard deviation (SD) of SBP, coefficient of variation (CV) of SBP, average real variability (ARV) of SBP, maximum and minimum values of the morning and evening average (MEave) of a period of SBP or a combination thereof. In a more preferred embodiment, the data of SBP comprise maximum and minimum values of MEave of a period of SBP. In another embodiment, the data of DBP comprise the mean value of DBP, SD of DBP, CV of DBP, ARV of DBP, maximum and minimum values of MEave of a period of DBP or a combination thereof. In some aspects, the MEave of SBP and DBP is collected by obtaining the blood pressure in the morning from 6 am to 8 am/in the evening from 4 pm to 6 pm before eating. In another embodiment, the collection period of SBP and DBP is from 1 to 12 weeks. In a preferred embodiment, the collection period of SBP and DBP is from 1 to 6 weeks. In a more preferred embodiment, the collection period of SBP and DBP is 1 week.

In one embodiment, the dataset further comprises temperature data and air pollution data. In a preferred embodiment, the air pollution data comprises air quality index (AQI), O₃, PM10, and PM2.5. In a more preferred embodiment, the air pollution data comprises PM2.5.

In the present invention, the dataset is randomly divided into a training set and a validation set for training the at least one machine learning model. Usually, the training set is used in conjunction with the validation set. The term “validation set” refers to a set of patients in a statistical sample, data of which subjects are used to validate or evaluate the quantitative values of interest determined using a training set.

In one embodiment, the at least one machine learning model comprises logistic regression (LR), deep neural networks (DNNs), random forest (RF), light gradient boosting machine (LightGBM), extreme gradient boosting (XGboost), decision trees (DTs), k nearest neighbor (KNN), adaboost, gradient boosting (Gboost), DT bagging (DTB), knn bagging (KNNB), or RF bagging (RFB). In a preferred embodiment, the at least one machine learning model comprises XGBoost, DTB, or RF. In a preferred embodiment, the first machine learning model comprises XGBoost, DTB, or RF.

In addition, the at least one machine learning model comprises the deep-learning architectures. In some aspects, the deep-learning architectures comprise deep neural networks, deep belief networks, deep reinforcement learning, recurrent neural networks, convolutional neural networks and transformers.

In the present invention, the at least one machine learning model may be a supervised machine learning algorithm. The supervised machine learning algorithm may be trained by using prior data of the patients, prior data of similar patients, or a combination thereof. The supervised machine learning algorithm may be a regression algorithm, a support vector machine, a decision tree, a neural network, or the like. In cases in which the machine learning model is the regression algorithm, the weights may be regression parameters. The supervised machine learning algorithm may be a binary classifier that predicts whether or not the patient will experience CVD event. The binary classifier may generate a probabilistic risk score between 0 and 1. In some cases, the system may map the probabilistic risk score to a qualitative risk category. Alternatively, the supervised machine learning algorithm may be a multi-class classifier that predicts a qualitative risk category directly. In addition, the internal validation of the at least one machine learning model is performed by using ten-fold cross-validation.

In another embodiment, the threshold value of accuracy is 0.85. In a preferred embodiment, the threshold value of accuracy is 0.9. In a more preferred embodiment, the threshold value of accuracy is 0.95.

In one embodiment, the data related to CVD onset time of the CVD patients comprise the CVD onset time of the CVD patients developing a CVD event within 1 month, 3 months, 6 months, 9 months, 1 year, 2 years, 3 years or 4 years. In some aspects, the at least one machine learning model is trained to analyze a relationship between the health data of the non-CVD patients and the CVD patients and the data related to CVD onset time of the CVD patients for predicting CVD occurrence in the future. Therefore, the present invention can use the data related to different CVD onset time of the CVD patients for training the at least one machine learning model to produce different prediction models, and the different prediction models are used for predicting a probability of a subject developing a CVD event at different time points in the future, in which the different time points are within a period of 1 month, 3 months, 6 months, 9 months, 1 year, 2 years, 3 years or 4 years.

In the present invention, the health data of the patients are used to establish a predicting model with higher accuracy for long-term (1 to 4 years) CVD occurrences. In addition, the health data of the patients, the temperature data and the air pollution data are used to establish a predicting model with higher accuracy for short-term (1 to 9 months) CVD occurrences. Therefore, the prediction model of the present invention can estimate onset risk of CVD at different time points. In one embodiment, the time points comprise 1 month, 3 months, 6 months, 9 months, 1 year, 2 years, 3 years or 4 years.

The present invention also provides a system comprising one or more computers and one or more storage devices for storing an operable program, when executed by the one or more computers, to enable the one or more computers to perform the following operations for producing a predicting model for estimating a risk of CVD, comprising: (a) obtaining a dataset from one or more sources, wherein the dataset comprises health data of non-CVD patients and CVD patients and data related to CVD onset time of the CVD patients, and the health data comprise demographic data, personal habits data, disease data, treatment data, blood analysis data, and blood pressure data; (b) inputting the dataset to at least one machine learning model for training the at least one machine learning model to predict CVD occurrence; (c) assessing accuracy of the at least one machine learning model from the step (b) and selecting a first machine learning model from the at least one machine learning model when the accuracy of the first machine learning model is higher than a threshold value of accuracy; and (d) using the first machine learning model to produce the prediction model for estimating a risk of CVD at different time points.

The present invention further provides a method for predicting a risk of CVD of a subject, which comprises: (i) obtaining health data of the subject, wherein the health data comprise demographic data, personal habits data, disease data, treatment data, blood analysis data, and blood pressure data; (ii) inputting the health data into the above predicting model for estimating a risk of CVD; and (iii) outputting a prediction result of the risk of CVD at different time points.)

In another embodiment, the prediction result of the prediction model for estimating a risk of CVD provides the probability of the subject developing a CVD event within a period of 10 years. In a preferred embodiment, the prediction result of the prediction model for estimating a risk of CVD provides the probability of the subject developing a CVD event within a period of 6 years. In a preferred embodiment, the prediction result of the prediction model for estimating a risk of CVD provides the probability of the subject developing a CVD event within a period of 4 years.

In one embodiment, the prediction result of the prediction model for estimating a risk of CVD provides the probability of the subject developing a CVD event within 1 month, 3 months, 6 months, 9 months, 1 year, 2 years, 3 years or 4 years.

In another embodiment, the method further comprises a step (iv) after the step (iii), wherein the step (iv) comprises determining whether or not a medical intervention is initiated for the subject having the risk of CVD based on the prediction result.

The present invention further provides a computer-implemented method of determining a risk of CVD of a subject, which comprises: receiving health data of the subject; using the above predicting model for estimating a risk of CVD to determine the risk of CVD of the subject based on the received health data; and outputting the determined risk of CVD at different time points, wherein the predicting model for estimating a risk of CVD assesses the determined risk of CVD by analyzing a relationship between the occurrence of CVD and the received health data.

In one embodiment, the subject and the patient are human.

The present invention also provides a healthcare system, comprising: (a) a patient monitoring module for collecting patient data generated by real-time monitoring of a patient; (b) a database for collecting health data of the patient, wherein the health data comprise demographic data, personal habits data, disease data, treatment data, blood analysis data, and blood pressure data; and (c) an integrated module for receiving the patient data and the health data, analyzing the patient data and the health data by using the above predicting model for estimating a risk of CVD, and outputting a prediction result of the risk of CVD of the patient at different time points based on the analysis of the predicting model for estimating a risk of CVD.

In one embodiment, the patient monitoring module is a remote patient monitoring module. Therefore, the remote patient monitoring module is able to collect patient data by using remote and real-time monitoring of the patient. In some aspects, the function of the patient monitoring module comprises vital sign captures. In addition, the purpose of the remote patient monitoring module is to monitor home blood pressure of the patient.

In another embodiment, the patient monitoring module, the database and the integrated module are connected with each other.

### EXAMPLES

The embodiment of the present invention could be implemented with different content and is not limited to the examples described in the following text. The following examples are merely representative of various aspects and features of the present invention.

### 1. Study Design

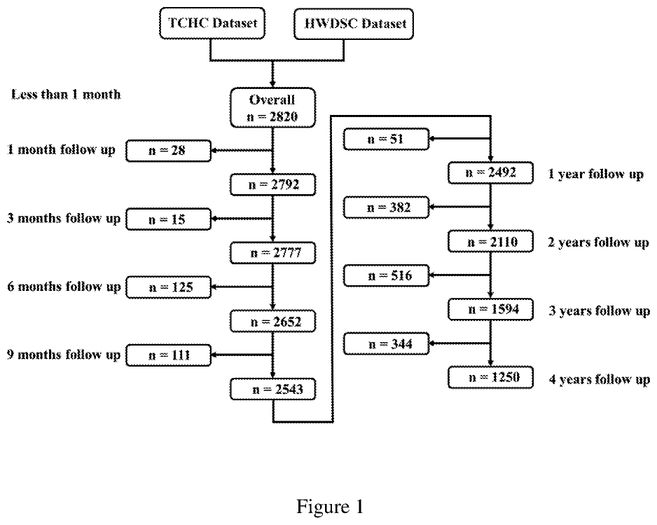

This model of the present invention was constructed by two datasets. One was collected by the Taiwan Consortium of Hypertension-associated Cardiac Disease (TCHC) dataset, which was included by 11 medical centers in Taiwan and is a nonprofit research alliance focusing on hypertension and hypertension-related disease clinical trials and research collaboration, and the other one was Taiwan Health and Welfare Data Science Center (HWDSC) database, which was contained with 2000 to 2017 registry for Taiwan National Health Insurance beneficiaries, Ambulatory Care Claims, Inpatient Claims, Pharmacies Dataset, Cause of Death Datasets. The combined dataset was with nearly 2820 participants (FIG. 1); roughly 80% and 20% of the data set were randomly selected and used for training and validation, respectively. Because CVD occurred in only 20% of patients in the data set, 80% of the CVD-positive and 80% of the CVD-negative data sets were used for training. The remaining 20% of the items were used for testing. TCHC contained cardiovascular disease records, blood pressure data, medication records, lifestyle interviews, basic health information, and other disease records. It was beneficial to understand not only the impact of measurable health values on cardiovascular disease, but also whether there was an interaction between different diseases. Several approaches were used: logistic regression (LR), deep neural networks (DNNs), random forest (RF), light gradient boosting machine (LightGBM), eXtreme gradient boosting (XGboost), decision trees (DTs), k nearest neighbor (KNN), Adaboost, gradient boosting (Gboost), DT bagging (DTB), knn bagging (KNNB), and RF bagging (RFB). Eventually, the present invention left the three models with the best performance: XGBoost, DTB, and RF.

The proportion of CVD outcome was shown as Table 1.

TABLE 1
Proportion of CVD outcome

| | 1 month | 3 months | 6 months | 9 months |
|---|---|---|---|---|
| CVD negative | 99.1% | 97.8% | 96.2% | 94.3% |
| CVD positive | 0.9% | 2.2% | 3.8% | 5.7% |
| | 1 year | 2 years | 3 years | 4 years |
| CVD negative | 92.6% | 87.7% | 83.2% | 79.5% |
| CVD positive | 7.4% | 12.3% | 16.8% | 20.5% |

### 2. Data Processing

The main features are as follows:

### 2-1. Non-BP-Related Features:

Demographics: age, gender, BMI, and waist circumference
Habits: smoking, alcohol-drinking, and exercise habits
Disease: hypertension (HT), diabetes mellitus (DM), and hyperlipidemia (HL)
Treatment: antihypertensive drugs (Anti-BP drugs), antidiabetic drugs, lipid-lowering drugs, and aspirin
Others: GOT, GPT, glucose (Glu_AC and Glu_PC), HbAlc, cholesterol, LDL, and HDL

### 2-2. BP-Related Features

Systolic blood pressure (SBP): Mean, standard deviation (SD), coefficient of variation (CV), average real variability (ARV), and maximum and minimum values of 7 day Morning and Evening average (MEave)] (morning during 6 am-8 am/evening during 4 pm-6 pm before eating.)

Diastolic blood pressure (DBP): Mean, SD, CV, ARV, and maximum and minimum values of MEave

### 2.3 Weather

Temperature (If the patient was diagnosed with CVD on a certain day, the temperature of that day was used. If the patient did not have CVD, the temperature of the day was used when blood pressure was measured.)

Air pollution values: including commonly used air pollution indicators such as AQI, O₃, PM10, and PM2.5.

AGE, gender, BMI, and waist circumference were easily attached and common features for the medical model. The habits (smoking, alcohol, exercise) were effective for one's health condition. The collected data related with the smoking, alcohol-drinking, and exercise habits was shown in Table 2. As the present invention mentioned, diseased may affect each other, so the present invention included hypertension, diabetes mellitus, hyperlipidemia, cancer, panic, anger, and blue as features. Glutamic oxaloacetic transaminase (GOT), serum glutamic pyruvic transaminase (GPT), and glucose were risk factors to health. Low-density lipoprotein (LDL), sometimes called “bad” cholesterol, and high-density lipoprotein (HDL), or called “good” cholesterol, were important parameters of cholesterol. The goal was to predict CVD occurrence by using BP data. However, BP data was complex. Therefore, the means of the SBP and DBP data were used instead. Some common CVD-related diseases were also analyzed. Age-based regression was used for missing data. Patients with and without CVD in the data set were labeled as 1 and 0, respectively. Similarly, patients with and without HT, DM, HL, and cancer were also labeled as 1 and 0, respectively. For numeric data, the original data were used. The present invention used the feature scaling method to standardize the influence of each feature. For categorical data, one-hot encoding was used to transform the features to be trainable. Tables 3 and 4 showed that continuous variables and binary variables were used in the present invention. In addition, the present invention used recursive feature elimination (RFE) based on logistic regression and principal component analysis (PCA) to perform feature selection.

TABLE 2
Categorical data of smoking, alcohol-drinking, and
exercise habits

| Ciga_Year | Alcohol_Year | Alcohol_% | Alcohol_ml | Sleep | Exercise |
|---|---|---|---|---|---|
| 1. less than | less than 1 year | 1. less than | 1. less than | 1. less than | 1. less than |
| 1 year | | 10% | 40 gm | 6 hours | once a |
| 2. 1-2 years | | (ex: beer) | 2. 41~120 gm | 2. 6~6.9 | month |
| 3. 2-3 years | | 2. 10-25% | 3. 121~200 gm | hours | 2. 2~3 times |
| 4. 3-4 years | | (ex: wine) | 4. more than | 3. 7~7.9 | a month |
| 5. 4-5 years | | 3. 25-40% | 200 gm | hours | 3. 1~2 times |
| 6. 5-6 years | | (ex: ginseng | | 4. more than | a week |
| 7. 6-7 years | | and antler | | 8 hours | 4. 3~4 times |
| 8. 7-8 years | | wine) | | | a week |
| 9. 8-9 years | | 4. more than | | | 5. more than |
| 10. 9-10 years | | 40% (ex: | | | 5 times a |
| 11. more | | whiskey, | | | week |
| than 10 years | | sorghum) | | | |
| 12. unknown | | | | | |

TABLE 3
Continuous variables

| | | | | | | |
|---|---|---|---|---|---|---|
| Age | Heart rate | Waist circumference | BMI | GOT | GPT | glu_AC |
| glu_PC | HbA1c | Total_Cholesterol | HDL | LDL | | |
| Mean_7d_DBP | Sd_7d_DBP | CV_7d_DBP | ARV_7d_DBP | VIM_7d_DBP | | |
| Mean_7d_SBP | Sd_7d_SBP | CV_7d_SBP | ARV_7d_SBP | VIM_7d_SBP | | |

TABLE 4
Binary variables

| | | | |
|---|---|---|---|
| CVD_before | hypertension_before | diabetes_before | hyperlipidemia_before |
| cancer_before | chronichepatis_before | Gender | |

Some important characteristics were defined as follows: the average of morning and evening value (MEave) BP values, the SD of the MEave of home SBP and DBP, the CV of the MEave of home SBP and DBP, the ARV of the MEave of home SBP and DBP, and the variability independent of the mean (VIM) of the MEave of home SBP and DBP. Despite the inclusion of BP data, features related to CVD were chosen to construct the model. The TCHC includes systolic and diastolic BP for each patient four times per day for seven days. Interpolation was used to reconstruct missing data. The method accurately identifies BP characteristics, variability, and trends.

After experimentation, it was found that most blood pressure-related parameters do not affect the accuracy of the model. Therefore, in Tables 5, 6 and 7, the present invention only used MEave of SBP as the prediction parameter to reduce the training load of the model.

### 3. Model

According to past experiments, the present invention used the three models with the highest accuracy to predict CVD occurrence. The present invention could get wonderful results when the present invention predicted short-term CVD occurrence. Otherwise, when predicting long-term CVD occurrence, the accuracy would fall around 0.85. However, the area under curve (AUC) was mostly very high, nearly 0.9.

4. Prediction of CVD with Temperature and Air-Pollution

Tables 5, 6, and 7 presented the results obtained by adding air temperature and air pollution to the above model. The calculation of temperature was the average temperature of the seven days when blood pressure was measured, and the calculation of air pollution was the average PM2.5 value of seven days when blood pressure was measured. It could be seen from Table 6 that after adding air temperature or air pollution, the accuracy of the model was slightly improved.

For temperature, only the temperature on the day when the cardiovascular disease occurred was used, as the effect of temperature was immediate.

TABLE 5
Model comparison with results for short-term CVD
occurrence with only temperature

| Model | Accuracy | Recall | Precision | F1 score | AUC |
|---|---|---|---|---|---|
| XGB_1m | 0.849 | 0.502 | 0.500 | 0.465 | 0.449 |
| RF_1m | 0.893 | 0.524 | 0.500 | 0.481 | 0.523 |
| DTB_1m | 0.880 | 0.543 | 0.502 | 0.479 | 0.536 |

It could be seen from Table 5 that using only temperature to predict the occurrence of cardiovascular disease could achieve an accuracy rate of over 0.88, which was already a good predictive model. However, more features could be added to improve the accuracy.

Since the additional features to be included involve basic life characteristics, blood pressure features, and air pollution, which were not only short-term factors but also common causes of cardiovascular disease in the medium to short term, the present invention expanded the dataset to include patients who had experienced cardiovascular disease within 1, 3, 6, and 9 months.

In Table 6, it could be observed that after incorporating other features, the model's accuracy had significantly increased, with an accuracy rate of up to 0.99 and an average of above 0.95. This could be considered a very accurate medium to short-term predictive model for cardiovascular disease.

TABLE 6
Model comparison with results for short-term CVD
occurrence with temperature and air-pollution

| Model | Accuracy | Recall | Precision | F1 score |
|---|---|---|---|---|
| XGB_1m | 0.99 | 0.557 | 0.621 | 0.576 |
| XGB_3m | 0.972 | 0.543 | 0.631 | 0.56 |
| XGB_6m | 0.959 | 0.601 | 0.696 | 0.626 |
| XGB_9m | 0.945 | 0.648 | 0.755 | 0.678 |
| XGB_1m_temp | 0.99 | 0.557 | 0.621 | 0.576 |
| XGB_3m_temp | 0.972 | 0.543 | 0.631 | 0.56 |
| XGB_6m_temp | 0.959 | 0.601 | 0.696 | 0.626 |
| XGB_9m_temp | 0.945 | 0.648 | 0.755 | 0.678 |
| XGB_1m_airpo | 0.991 | 0.516 | 0.546 | 0.523 |
| XGB_3m_airpo | 0.976 | 0.553 | 0.715 | 0.577 |
| XGB_6m_airpo | 0.956 | 0.533 | 0.624 | 0.544 |
| XGB_9m_airpo | 0.942 | 0.607 | 0.718 | 0.637 |

### 5. Long-Term Model

The medium to short-term model had shown good results. Next, the present invention wanted to attempt long-term prediction of cardiovascular disease. The present invention set the time frame for patients who had experienced cardiovascular disease within 1, 2, 3, and 4 years. Since there were more long-term patients and more variables to consider, the present invention expected a decrease in accuracy rate.

As shown in Table 7, except for the prediction of cardiovascular disease within four years which was around 0.88, the models for 1, 2, and 3 years could achieve an accuracy rate of above 0.9. Compared to the medium to short-term model, although it was slightly inferior, it was still a model with good accuracy rate.

TABLE 7
Model comparison with results for long-term CVD occurrence

| Model | accuracy | recall | precision | f1 | AUC |
|---|---|---|---|---|---|
| XGB_1y | 0.941 | 0.693 | 0.815 | 0.735 | 0.857 |
| RF_1y | 0.94 | 0.671 | 0.82 | 0.717 | 0.904 |
| DTB_1y | 0.933 | 0.674 | 0.771 | 0.707 | 0.876 |
| XGB_2y | 0.922 | 0.76 | 0.849 | 0.793 | 0.897 |
| RF_2y | 0.930 | 0.747 | 0.901 | 0.798 | 0.928 |
| DTB_2y | 0.922 | 0.758 | 0.85 | 0.793 | 0.908 |
| XGB_3y | 0.91 | 0.798 | 0.863 | 0.823 | 0.909 |
| RF_3y | 0.915 | 0.775 | 0.904 | 0.819 | 0.944 |
| DTB_3y | 0.906 | 0.792 | 0.854 | 0.815 | 0.921 |
| XGB_4y | 0.88 | 0.798 | 0.827 | 0.806 | 0.887 |
| RF_4y | 0.881 | 0.753 | 0.853 | 0.787 | 0.916 |
| DTB_4y | 0.862 | 0.75 | 0.806 | 0.77 | 0.894 |

The results in the present invention were also validated by categorical variables screening, such as the relationship between size, exercise, alcohol consumption, and medication with CVD. Moreover, it was not easy to achieve high accuracy for traditional regression models, such as LR. On the contrary, machine learning models were suitable for dealing with the biological binary judgment model. The experiments had several suitable models for predicting CVD risks, such as RF, DTB, and xGBoost. The goal of the present invention was to identify CVD risks. However, the BP records were for only seven days based on the limited dataset size; records for an extended period of 4-6 weeks might yield more accurate results.

In conclusion, the people participating in the present invention were divided into short-term (within 1/3/6/9 months) and long-term (within 1/2/3/4 years) groups based on the occurrence of CVD. Depending on the different time periods, the input factors would also be different.

For the short-term model: a combination of basic physical characteristics, blood pressure data and temperature is used for predicting short-term CVD occurrence.

For the long-term model: basic physical characteristics is used for predicting long-term CVD occurrence.

The model constructed in this present invention could be applied to predicting short-term acute cardiovascular diseases, as well as serving as a predictive model for long-term follow-up observation and preventive medicine.

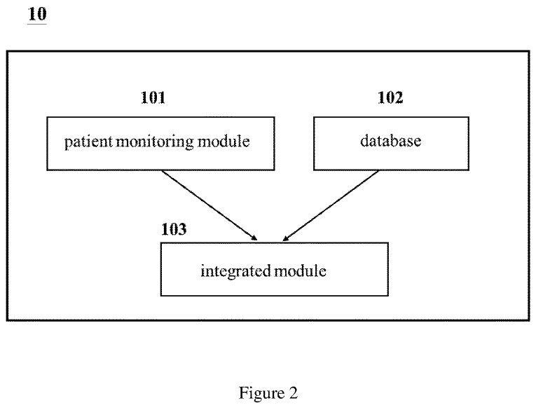

In addition, the present invention further provided a healthcare system for estimating a risk of cardiovascular disease of a patient. In FIG. 2, the healthcare system 10 comprised: (a) a patient monitoring module 101 for collecting patient data generated by real-time monitoring of the patient; (b) a database 102 for collecting health data of the patient, wherein the health data comprise demographic data, personal habits data, disease data, treatment data, blood analysis data, and blood pressure data; and (c) an integrated module 103 for receiving the patient data and the health data, analyzing the patient data and the health data by using the above predicting model for estimating a risk of CVD, and outputting a prediction result of the risk of CVD of the patient at different time points based on the analysis of the predicting model for estimating a risk of CVD. In this healthcare system, the patient monitoring module was a remote patient monitoring module.

Those skilled in the art recognize the foregoing outline as a description of the method for communicating hosted application information. The skilled artisan will recognize that these are illustrative only and that many equivalents are possible.

What is claimed is:

1. A method for producing a predicting model for estimating a risk of cardiovascular disease (CVD), which comprises: (a) obtaining a dataset from one or more sources, wherein the dataset comprises health data of non-CVD patients and CVD patients and data related to CVD onset time of the CVD patients, and the health data comprise demographic data, personal habits data, disease data, treatment data, blood analysis data, and blood pressure data; (b) inputting the dataset to at least one machine learning model for training the at least one machine learning model to predict CVD occurrence; (c) assessing accuracy of the at least one machine learning model from the step (b) and selecting a first machine learning model from the at least one machine learning model when the accuracy of the first machine learning model is higher than a threshold value of accuracy; and (d) using the first machine learning model to produce the prediction model for estimating a risk of CVD at different time points.

2. The method of claim 1, wherein the CVD comprises myocardial infarction, stroke, heart failure, cardiovascular death or a combination thereof.

3. The method of claim 1, wherein the demographic data comprise age, gender, body mass index (BMI), and waist circumference.

4. The method of claim 1, wherein the personal habits data comprise smoking, alcohol-drinking, exercising habits or a combination thereof.

5. The method of claim 1, wherein the disease data comprise hypertension, diabetes mellitus, hyperlipidemia or a combination thereof.

6. The method of claim 1, wherein the treatment data comprise uses of antihypertensive drugs, antidiabetic drugs, lipid-lowering drugs, aspirin or a combination thereof.

7. The method of claim 1, wherein the blood analysis data comprise glutamic oxaloacetic transaminase (GOT), glutamic pyruvic transaminase (GPT), blood glucose, glycated hemoglobin, cholesterol, low-density lipoprotein (LDL), high-density lipoprotein (HDL) or a combination thereof.

8. The method of claim 1, wherein the dataset further comprises temperature data and air pollution data.

9. The method of claim 1, wherein the at least one machine learning model comprises extreme gradient boosting (XGboost), decision trees bagging (DTB), or random forest (RF).

10. The method of claim 1, wherein the time points comprise 1 month, 3 months, 6 months, 9 months, 1 year, 2 years, 3 years or 4 years.

11. A method for predicting a risk of cardiovascular disease (CVD) of a subject, which comprises: (i) obtaining health data of the subject, wherein the health data comprise demographic data, personal habits data, disease data, treatment data, blood analysis data, and blood pressure data; (ii) inputting the health data into the predicting model for estimating a risk of CVD produced by the method of claim 1; and (iii) outputting a prediction result of the risk of CVD of the subject at different time points.

12. The method of claim 11, which further comprises a step (iv) after the step (iii), wherein the step (iv) comprises determining whether or not a medical intervention is initiated for the subject having the risk of CVD based on the prediction result.

13. A healthcare system comprising: (a) a patient monitoring module for collecting patient data generated by real-time monitoring of a patient; (b) a database for collecting health data of the patient, wherein the health data comprise demographic data, personal habits data, disease data, treatment data, blood analysis data, and blood pressure data; and (c) an integrated module for receiving the patient data and the health data, analyzing the patient data and the health data by using the predicting model for estimating a risk of cardiovascular disease (CVD) produced by the method of claim 1, and outputting a prediction result of the risk of CVD of the patient at different time points based on the analysis of the predicting model for estimating a risk of CVD.

14. The healthcare system of claim 13, wherein the patient monitoring module is a remote patient monitoring module.
